SEQUENCE LISTING (18 sequences)

SEQ ID NO: 1 - DNA, 19 bp, Artificial Sequence
  Single strand DNA oligonucleotide
ctgcacttccttcccgaaa

SEQ ID NO: 2 - DNA, 19 bp, Artificial Sequence
  Single strand DNA oligonucleotide
cggctgaaaggtggtactt

SEQ ID NO: 3 - DNA, 24 bp, Artificial Sequence
  Single strand DNA oligonucleotide
  misc_feature (1)..(1): Biotin + Alexa 532 5′ conjugated
  misc_feature (9)..(10): N,N-diethyl-4-(4-nitronaphthalen-1-ylazo)- phenylamine (ZEN) quencher is connected via phosphate linkages to the ribose backbone of the oligonucleotide
  misc_feature (24)..(24): Iowa Black FQ  3′ conjugated
cgcttcactcacaaagcacgcatt

SEQ ID NO: 4 - DNA, 717 bp, Gallus gallus
ctcgagaaataccacttttctctcgaaaatcatgcattttcatccaaaaataccacctgtctcccaaaaattctgcacttccttcccgaaaataccacttttcgctgggaaataacacatttctaccccaaatataacacgcttcactcacaaagcacgcattttcaccccgaaagtaccacctttcagccgaaaattacgctttttcctccagaaaataccacttctcaaacagaaatatcacgtttcgccaagaaaatagcaccattcaccccaaaatcacgcgttttctctccagaactaccacttttctcacggaaatcacacattttcttcccgaaagtaccaccttgcacacgaaaatcacgcattttctgcgcgaaacaaccccatttcaccccaaaatcagtctttttcttccggaaaatacaacttttctaacgaaatccatgcgttatcactctgaaaatacacgtttttgcccgaaaatcacgcattttcccttcgtaaattccccatgtcgccagaaaataattcatttccttaccgtaaatgccccttttcacccaaaaatcacgcatttccccccggaaaaacgcacctctgtccaaacatcacgcattttctacccgaaaataccacttttggctgggaaataacacatttctcccccaaatataccacctttcaccccaaaatcacgcattttctctcgag

SEQ ID NO: 5 - DNA, 1211 bp, Gallus gallus
aattctcttcgaaaaaaacgcttttgcctgaaaatcacgcgttttctacccaaaaataccaatattttgcccggaaatcacgcatttacacctcaaaaacgtcacttttcgcccaaaaagcaagcattttctcttagaaaattttacccccaaaacacgcatttttaccataaaaataccatttttcgcacgagaatcatgtatgttcagttcaaaactactgcatttcaccccaaaatcacgctttttccctccagaaatgcaactttttaaatagaaatcacgcgtttcctccacgaaaataccgcctttcaccccaaaatcacgcattttctctccaaaaataccacctttctctcgaaaagcacgcatttccacacggaaaataccacttttcccttgaaaattcccacattttcttctcgaaaataccacttctcaccggaaaataacgcctttctaccgcaaatacaccacgtttcaccccaaaatcacgcattttcaccccgaaaataccgcctttcaccccaaaatcacgcattttcttcccgaaaataccacctttcaaaggaaaatcacttcattttctccccgaaaataccacctttcccccgaaaatcacgcattttctgcgcgaagcaaccccatttctcccaagaaccacgcatttttttcacgaaaattgtacgttcaaaggaaaatcgcacattttctccctgaacataccacctttcacccaaaaattccgcgttttccgcgtgaaggaaccccgcttcacacgaatatcacgcattgtctaccggaaaataccacttttctaacgaaaatcacgcactgtcccgccgaaaacacaagttttcgcccgaaaatcacgcattttcccccagaaaaaaagcacctctgtccacaagtcacgcatcttgtacccgaaaataccacttttctcctgaaaatcccgcattttcaccccacaaatgccacttttcgcgcgaaaagcatttccttcgggaaaatcaccctcttcaaccgaaaaacacgtgttttctccttgaaaataccatctttcgcctgagaatcacgcatgttcacttcgaaaataccccatttccccagaaaatctcgcactgtctcctcgaaaataccctgagcgcaggtccgcgcatgcgcagtgcgatcgcaaatccacgcatacgcggtgctccccctgcgag

SEQ ID NO: 6 - DNA, 22 bp, Artificial Sequence
  Single strand DNA oligonucleotide
ctccctgaacataccacctttc

SEQ ID NO: 7 - DNA, 24 bp, Artificial Sequence
  Single strand DNA oligonucleotide
cccgcttcacacgaatatcacgca

SEQ ID NO: 8 - DNA, 18 bp, Artificial Sequence
  Single strand DNA oligonucleotide
cgggacagtgcgtgattt

SEQ ID NO: 9 - DNA, 26 bp, Artificial sequence
  Single strand DNA oligonucleotide
acaggtacgttaatagttaatagcgt

SEQ ID NO: 10 - DNA, 22 bp, Artificial sequence
  Single strand DNA oligonucleotide
atattgcagcagtacgcacaca

SEQ ID NO: 11 - DNA, 26 bp, Artificial sequence
  Single strand DNA oligonucleotide
  misc_feature (1)..(1): Biotin + Atto532   5′ conjugated
  misc_feature (9)..(10): N,N-diethyl-4-(4-nitronaphthalen-1-ylazo)- phenylamine (ZEN) quencher is connected via phosphate linkages to the ribose backbone of the oligonucleotide
  misc_feature (26)..(26): Iowa Black FQ  3′ conjugated
acactagccatccttactgcgcttcg

SEQ ID NO: 12 - DNA, 22 bp, Artificial sequence
  Single strand DNA oligonucleotide
gtgaratggtcatgtgtggcgg

SEQ ID NO: 13 - DNA, 26 bp, Artificial sequence
  Single strand DNA oligonucleotide
caratgttaaasacactattagcata

SEQ ID NO: 14 - DNA, 25 bp, Artificial sequence
  Single strand DNA oligonucleotide
  misc_feature (1)..(1): Biotin + Atto532  5′ conjugated
  misc_feature (9)..(10): N,N-dAtto532iethyl-4-(4-nitronaphthalen-1- ylazo)-phenylamine (ZEN) quencher is connected via phosphate linkages to the ribose backbone of the oligonucleotide
  misc_feature (25)..(25): Iowa Black FQ  3′ conjugated
caggtggaacctcatcaggagatgc

SEQ ID NO: 15 - DNA, 25 bp, Artificial sequence
  Single strand DNA oligonucleotide
caggtggaacctcatcaggagatgc

SEQ ID NO: 16 - DNA, 26 bp, Artificial sequence
  Single strand DNA oligonucleotide
acactagccatccttactgcgcttcg

SEQ ID NO: 17 - DNA, 228 bp, Artificial sequence
  E-gene of SARS-CoV2
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 18 - DNA, 29839 bp, Artificial sequence
  Rdrp gene target nucleic acid sequence
aaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgattttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattactccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctttcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcagcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaataaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgccgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcctggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

(19) United States
(12) Patent Application Publication
(10) Pub. No.: US 2023/0257832 A1
(43) Pub. Date: Aug. 17, 2023

(54) DETECTING AND QUANTIFYING A VIRAL TARGET NUCLEIC ACID SEQUENCE

(71) Applicant: Bar-Ilan University, Ramat-Gan (IL)

(72) Inventors: Amos DANIELLI, Moshav Hadar-Am (IL); Michael MARGULIS, Shoham (IL)

(73) Assignee: Bar-Ilan University, Ramat-Gan (IL)

(21) Appl. No.: 18/014,507

(22) PCT Filed: Jul. 7, 2020
(86) PCT No.: PCT/IL2020/050761

§ 371 Date: Jan. 5, 2023

(51) Int. Cl. C12Q 1/70; C12Q 1/6851

(52) U.S. Cl. CPC C12Q 1/701; C12Q 1/6851; C12Q 1/6823

(57) ABSTRACT

Oligonucleotide probes for detecting target nucleic acid sequences are disclosed.

### SEQUENCE LISTING STATEMENT

The ASCII file, entitled 83275 Sequence Listing.txt, created on 7 Jul. 2020, comprising 46,463 bytes, submitted concurrently with the filing of this application is incorporated herein by reference.

### FIELD AND BACKGROUND OF THE INVENTION

The present invention, in some embodiments thereof, relates to compositions and methods for detecting and quantifying a target nucleic acid sequence.

Techniques for polynucleotide detection and quantification have found widespread use in basic research, diagnostics, and forensics. Polynucleotide detection can be accomplished by a number of quantitative and qualitative methods. Many methods rely on the use of the polymerase chain reaction (PCR) to amplify the amount of target nucleic acid sequence and the detection of fluorescence to monitor the amplification.

The TaqMan™ assay is one of such assays for quantifying polynucleotides (see e.g., U.S. Pat. No. 5,723,591). In a typical TaqMan™ assay, two PCR primers flank a central oligonucleotide probe. The probe contains a fluorophore and a quencher moiety that are in close proximity to each other. This allows the fluorescence energy from the fluorophore to be transferred to the quencher and become undetectable. During the polymerization step of the PCR, the polymerase cleaves the oligonucleotide probe. The cleavage causes the fluorophore and the quencher moiety to become physically separated, and thus allows the fluorescent emission from the fluorophore to be detected. As more PCR product is created, the intensity of the fluorescent emission increases. A defined signal threshold is determined for reactions comprising the target nucleic acid and reactions comprising the reference nucleic acid and the number of cycles required to reach this threshold value (Ct) is determined. The absolute or relative copy numbers of the target molecule can be determined on the basis of the Ct values obtained for the target nucleic acid and the reference nucleic acid. In another approach, the amount of the target molecule is determined by comparing the signal with a calibration curve.

Methods of improving the sensitivity of FRET-based assays (e.g., the Taqman™ assay) are required.

Background art includes U.S. Pat. Nos. 9,575,068, 8,465,989, US Application No. 2012-0135873-A1, Granevitze, Z., et al., Journal of Heredity, 2007. 98(3): p. 238-242, and Danielli et al., Opt Express, 2008. 16(23): p. 19253-9; Griffiths, R., et al., Mol Ecol, 1998. 7(8): p. 1071-5; Kalina, J., et al., Czech Journal of Animal Science, 2012. 57(4): p. 187-192; Rosenthal, N. F., et al., Poult Sci, 2010. 89(7): p. 1451-6; Danielli et al., Current Pharmaceutical Biotechnology, 11, 128-137 (2010) and Danielli et al., Biosensors and Bioelectronics, 25, 858-863 (2009); Margulis and Danielli et al., ACS Omega 2019, 4, 11749-11755.

### SUMMARY OF THE INVENTION

According to an aspect of the present invention there is provided a method of diagnosing a disease associated with a coronavirus infection in a subject comprising:
(a) contacting a sample of the subject with:
   (i) a pair of amplification primers, the primers having a sequence such that they are capable of amplifying a target sequence which is specific to the coronavirus;
   (ii) a polymerase enzyme having 5′ nuclease activity; and
   (iii) an oligonucleotide probe, wherein the oligonucleotide probe is between 10-300 nucleotides in length and comprises a fluorescent moiety attached to a first nucleotide of the probe, a first quencher moiety attached to a second nucleotide of the probe, and a second quencher moiety attached to a third nucleotide which is between the first and the second nucleotide, wherein the oligonucleotide probe anneals within the target sequence bound by the amplification primers,
wherein the contacting is effected under conditions that allow extension of the amplification primers and which allow the 5′ nuclease activity of the polymerase enzyme to cleave the annealed oligonucleotide probe so as to generate a cleaved fluorescent product; and
(b) detecting fluorescence of the cleaved fluorescent product, wherein the presence of the cleaved fluorescent product is indicative of the subject having a disease associated with the coronavirus.

According to an aspect of the present invention there is provided a kit for detecting SARS-CoV2 virus comprising:
(i) a probe having a nucleic acid sequence as set forth in SEQ ID NO: 16 and primers having a nucleic acid sequence as set forth in SEQ ID NOs: 9 and 10; or
(ii) a probe having a nucleic acid sequence as set forth in SEQ ID NO: 15 and primers having a nucleic acid sequence as set forth in SEQ ID NOs: 12 and 13.

According to an aspect of the present invention there is provided a method of determining the sex of a chicken comprising:
(a) contacting DNA of the chicken with:
   (i) a pair of amplification primers, the primers having a sequence such that they are capable of amplifying a target sequence on a W chromosome that repeats at least 10 times;
   (ii) a polymerase enzyme having 5′ nuclease activity; and
   (iii) an oligonucleotide probe, wherein the oligonucleotide probe is between 10-300 nucleotides in length and comprises a fluorescent moiety attached to a first nucleotide of the probe, a first quencher moiety attached to a second nucleotide of the probe, and a second quencher moiety attached to a third nucleotide which is between the first and the second nucleotide, wherein the oligonucleotide probe anneals within the target sequence bound by the amplification primers,
wherein the contacting is effected under conditions that allow extension of the amplification primers and which allow the 5′ nuclease activity of the polymerase enzyme to cleave the annealed oligonucleotide probe so as to generate a cleaved fluorescent product; and
(b) detecting fluorescence of the cleaved fluorescent product.

According to an aspect of the present invention there is provided a method of detecting a target nucleic acid sequence in a DNA sample comprising:
(a) contacting the sample with a pair of amplification primers, a polymerase enzyme having 5′ nuclease activity and the oligonucleotide of any one of claims 29-41, wherein the oligonucleotide probe anneals within the target nucleic acid sequence bound by the amplification primers, wherein the contacting is effected under conditions that allow extension of the amplification primers and further which allow the 5′ nuclease activity of the polymerase enzyme to cleave the annealed oligonucleotide so as to generate a cleaved fluorescent product;
(b) immobilizing the cleaved fluorescent product via a second member of the affinity pair; and
(c) detecting fluorescence of the cleaved fluorescent product.

According to an aspect of the present invention there is provided a oligonucleotide probe being 10-300 nucleotides in length, the probe comprising:
(i) a fluorescent moiety attached to a first nucleotide of the probe;
(ii) a first quencher moiety attached to a second nucleotide of the probe;
(iii) a second quencher moiety attached to a third nucleotide which is between the first and the second nucleotide; and
(iv) a first member of an affinity pair attached to the first nucleotide or to a nucleotide that is 5′ to the third nucleotide.

According to an aspect of the present invention there is provided a kit comprising the oligonucleotide of any one of claims 29-41 and a second member of the affinity pair which is attached to a solid support.

According to an aspect of the present invention there is provided a kit comprising the oligonucleotide of any one of claims 29-41 and a second member of the affinity pair which is attached to a solid support.

According to embodiments of the present invention, the oligonucleotide probe further comprises a first member of an affinity pair attached to the 5′ nucleotide.

According to embodiments of the present invention, the first nucleotide of the probe is the 5′ nucleotide.

According to embodiments of the present invention, the second nucleotide of the probe is the 3′ nucleotide.

According to embodiments of the present invention, the third nucleotide is 5-20 nucleotides from the 5′ nucleotide.

According to embodiments of the present invention, the method further comprises immobilizing the cleaved fluorescent product via a second member of the affinity pair following the contacting and prior to the detecting.

According to embodiments of the present invention, the immobilizing serves to concentrate the cleaved fluorescent product.

According to embodiments of the present invention, the detecting is effected within 40 minutes of the contacting.

According to embodiments of the present invention, the coronavirus is severe acute respiratory syndrome coronavirus 1 (SARS-CoV-1), severe acute respiratory syndrome coronavirus 2 (SARS-CoV-2) or Middle East respiratory syndrome coronavirus (MERS-CoV).

According to embodiments of the present invention, the coronavirus is severe acute respiratory syndrome coronavirus 2 (SARS-CoV-2).

According to embodiments of the present invention, the target sequence is comprised in the E_Sarbeco gene (E-gene) of the SARS-CoV2 virus.

According to embodiments of the present invention, the nucleic acid sequence of the probe is as set forth in SEQ ID NO: 16.

According to embodiments of the present invention, the nucleic acid sequence of the primers are as set forth in SEQ ID NOs: 9 and 10.

According to embodiments of the present invention, the target sequence is comprised in the RdRp gene of the SARS-CoV2 virus.

According to embodiments of the present invention, the nucleic acid sequence of the probe is as set forth in SEQ ID NO: 15.

According to embodiments of the present invention, the nucleic acid sequence of the primers are as set forth in SEQ ID NOs: 12 and 13.

According to embodiments of the present invention, the sample is derived from saliva, a nasal nasopharyngeal (NP) specimen, an oropharyngeal (OP) specimen, a nasal mid-turbinate swab, an anterior nares specimen, stool, sample or a nasopharyngeal wash.

According to embodiments of the present invention, the sample of the subject comprises cDNA which has been reverse transcribed from RNA present in the sample.

According to embodiments of the present invention, the kit further comprises a reverse transcriptase enzyme.

According to embodiments of the present invention, the oligonucleotide probe further comprises a first member of an affinity pair attached to the 5′ nucleotide.

According to embodiments of the present invention, the method further comprises immobilizing the cleaved fluorescent product via a second member of the affinity pair following the contacting and prior to the detecting.

According to embodiments of the present invention, the immobilizing serves to concentrate the cleaved fluorescent product.

According to embodiments of the present invention, the detecting is effected within 20 minutes of the contacting.

According to embodiments of the present invention, the fluorescent moiety is selected from the group consisting of an Atto dye, fluorescein, fluorescein chlorotriazinyl, rhodamine green, rhodamine red, tetramethylrhodamine, FITC, Oregon green, Alexa Fluor, FAM, JOE, ROX, HEX, Texas Red, TET, TRITC, TAMRA, cyanine-based dye and thiadicarbocyanine dye.

According to embodiments of the present invention, the first quencher moiety is selected from the group consisting of Dabcyl, TAMRA, Eclipse, DDQ, QSY, Blackberry Quencher, Black Hole Quencher, Qxl, Iowa black FQ, Iowa black RQ, and IRDye QC-1.

According to embodiments of the present invention, the second quencher moiety is a ZEN™ quencher or a TAO™ quencher.

According to embodiments of the present invention, the affinity pair comprises a biotin/avidin affinity pair or a biotin/streptavidin affinity pair.

According to embodiments of the present invention, the oligonucleotide is no longer than 30 nucleotides.

According to embodiments of the present invention, the oligonucleotide has a sequence such that it hybridizes to a nucleic acid sequence that repeats more than 100 times in a single chromosome.

According to embodiments of the present invention, the chromosome is a W chromosome of a chicken.

According to embodiments of the present invention, the oligonucleotide comprises a nucleic acid sequence as set forth in SEQ ID NOs: 15 or 16.

According to embodiments of the present invention, the oligonucleotide comprises a nucleic acid sequence as set forth in SEQ ID NOs: 11 or 14.

According to embodiments of the present invention, the second member of the affinity pair is attached to a solid support.

According to embodiments of the present invention, the solid support comprises a magnetic bead.

According to embodiments of the present invention, the method further comprises using a magnetic field to concentrate the cleaved fluorescent product prior to the detecting.

According to embodiments of the present invention, the detecting is effected within 20 minutes of said contacting.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration of one embodiment of a magnetic modulation biosensing system setup. The 532 nm laser beam is reshaped using two plano-convex lenses (not shown), reflected by a dichroic mirror, and focused by an objective lens onto the sample of magnetic beads aggregated inside a borosilicate cuvette. The cuvette is positioned between two electromagnets that cause the beads to move in periodic motion, in and out of the laser beam. The laser excites the fluorophores in the sample and the emitted fluorescence is collected by the same objective lens, filtered by an emission filter and detected by a camera. Set of two emission filters block the non-specific and scattered light from reaching the camera.

FIG. 2 is a graph illustrating the detection of female-specific, non-repetitive target using regular Tagman™ probes. 50 ng of the genomic DNA were used in order to discriminate between male and female samples using regular Tagman™ probes and the MMB as detection system. Samples were subjected to 30 cycles of PCR and tested using the MMB system. All results are an average of three independent MMB measurements (n=3).

FIG. 3 is a graph illustrating the detection of PCR-amplified, female-specific, non-repetitive target using regular and double quenched (ZEN) Tagman™ probes. 361 bp DNA fragment (female-specific, non-repetitive sequence) was amplified by PCR. 43 ng of the amplified target were used to compare between regular and ZEN Tagman™ probes. Samples were subjected to 10 cycles of PCR and tested using the MMB system. All results are an average of three independent MMB measurements (n=3).

FIG. 4 is a graph that illustrates the minimal number of PCR cycles required for sex determination using XhoI repetitive sequence as a target, double quenched (ZEN) Tagman™ probes and MMB system as the detection method. 50 ng of genomic DNA were used with ZEN TaqMan probes. Samples were subjected to 5, 10, 20 and 30 cycles of PCR respectively and tested using the MMB system. All presented results are an average of three independent MMB measurements (n=3).

FIG. 5 is a graph that illustrates the minimal number of PCR cycles required for sex determination using XhoI repetitive sequence as a target, double quenched (ZEN) Tagman™ probes and real-time PCR as the detection method. 50 ng of genomic DNA were used with ZEN Tagman™ probes. Samples were subjected to 30 cycles of Real-Time PCR. Presented results are based on measurements of five independent samples (n=5).

FIG. 6 is a graph demonstrating detection of a synthetic SARS-CoV2 target using the MMB-based method. SARS-CoV2-negative RNA extracts were spiked with 10, 100, and 1000 copies of a synthetic E-gene DNA target. After 35 cycles, clear distinction could be made between the positive and the negative samples, where even at the lowest contamination level of 10 copies, the signal of the experimental sample was ˜3 times higher compared with the negative RNA sample.

FIG. 7 is a graph illustrating E-gene based testing of clinical samples of healthy and SARS-CoV2 infected individuals. Nine negative and 32 positive samples (confirmed positive and negative using real time PCR) were tested. According to preliminary results, the MMB-based E-gene detection method allows 100% sensitivity and 100% specificity, which is at least as sensitive and specific as the gold standard RT-qPCR methodology, but requires 35 minutes from RNA sample to results, compared to 1.5-2 hours requirement of the conventional method.

FIG. 8 is a graph illustrating RdRp2 gene-based testing of clinical samples of healthy and SARS-CoV2 infected individuals. Ten negative and 32 positive samples (confirmed positive and negative using real time PCR) were tested. According to the presented results, the MMB-based RdRp-gene detection method allows 96.9% sensitivity and 100% specificity, which is on par with the sensitivity and specificity of the gold standard RT-qPCR methodology, but requires 35 minutes from RNA sample to results, compared to 1.5-2 hours requirement of the conventional method.

FIGS. 9A-B are graphs illustrating the correlation between the Ct of samples that were identified using qPCR and the signal generated by the MMB system using a) an RdRp2 probe, and b) an E-gene probe.

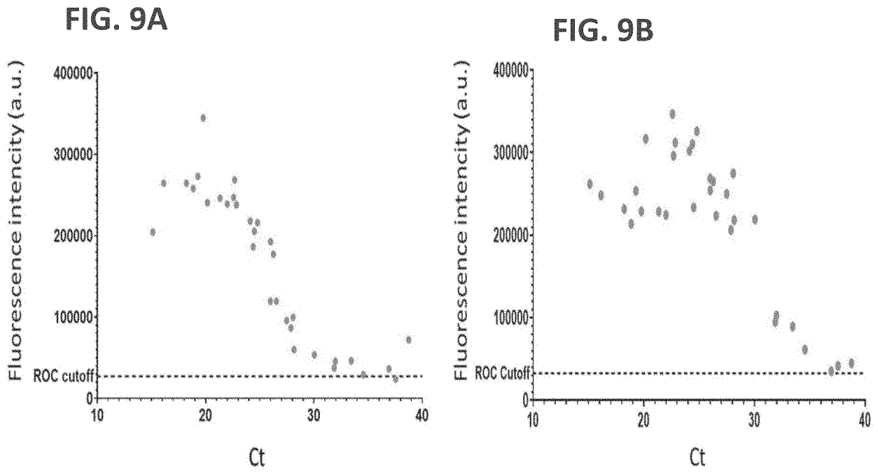

FIG. 10 is a graph illustrating the use of saliva as a source of the SARS-CoV-2 testing sample. Three samples from healthy patients were tested and compared with the same samples spiked with 10 and 100 copies of a synthetic E-gene target RNA. In addition, 10 and 100 copies of the synthetic E-gene target RNA were spiked in water (Ultra Pure H₂O) and compared with the saliva-based samples. All the samples were tested by the MMB system and the signal was normalized to the signal generated by a negative H₂O only sample.

FIG. 11 is a graph illustrating spiking of two samples (one of H₂O only and the other of a healthy patient saliva, 4 μL from each sample) with 1 μL of RNA extract from a positive patient (confirmed positive using real time PCR). Both spiked samples showed comparable signal to the purified RNA extract sample.

### DESCRIPTION OF SPECIFIC EMBODIMENTS OF THE INVENTION

The present invention, in some embodiments thereof, relates to compositions and methods for detecting and quantifying a target nucleic acid sequence.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In 2017, egg production in the United States exceeded 105 billion units, laid by more than 374 million laying hens. Hen populations requires constant maintenance, since “spent” hens of 70 weeks and older are unable to maintain egg production at the required level and are constantly being replaced by newly hatched chicks. Similar numbers of male chicks that have no commercial value for the industry are killed at the day of hatching each year, raising serious ethical questions.

PCR methods for in-ovo chick sexing have been described previously [Griffiths, R., et al., Mol. Ecol, 1998. 7(8): p. 1071-5; Kalina, J., et al., Czech Journal of Animal Science, 2012. 57(4): p. 187-192; and Rosenthal, N. F., et al., Poult. Sci, 2010. 89(7): p. 1451-6], but in order to use PCR in industrial-scale hatcheries the test times have to be significantly reduced.

SARS-Cov2 has rapidly spread and despite containment efforts, has caused a worldwide pandemic. A combination of fast contagion rate, high population density, and high volume of international travel in and out of the affected area facilitated the dissemination of the disease and required swift and accurate response on behalf of the healthcare authorities worldwide. One of the factors that had a significant impact on the effectiveness (or lack thereof) of the containment efforts was the ability to rapidly screen large populations for the presence of the infection. Unfortunately, most of the tests were (and still are) performed using the conventional RT-qPCR method. Relatively low throughput of this method presents a significant challenge for the containment and prevention efforts worldwide.

In the vast majority of the routine clinical tests for detecting SARS-CoV2 infection, 45 PCR cycles are performed, and, in many cases, the signal reaches the detectable level (Ct) only after the 37^{th} cycle. In general, PCR is usually limited to 35 cycles because of the tendency of the reaction towards false-positive results at the higher numbers of amplification cycles. A high number of PCR cycles required for detection of SARS-CoV2 infection leads to a high probability of false positive results, reducing the specificity of the test. For that reason, any reduction of the number of PCR cycles required for the diagnosis of the infection directly contributes to assays' specificity but may have a negative impact on its sensitivity.

To address the above issues, the present inventors propose a modified and improved molecular approach, based on principles of a FRET-based (e.g., TaqMan™) assay and magnetic and optical-based detection methods, such as the Magnetic Modulation Biosensing (MMB).

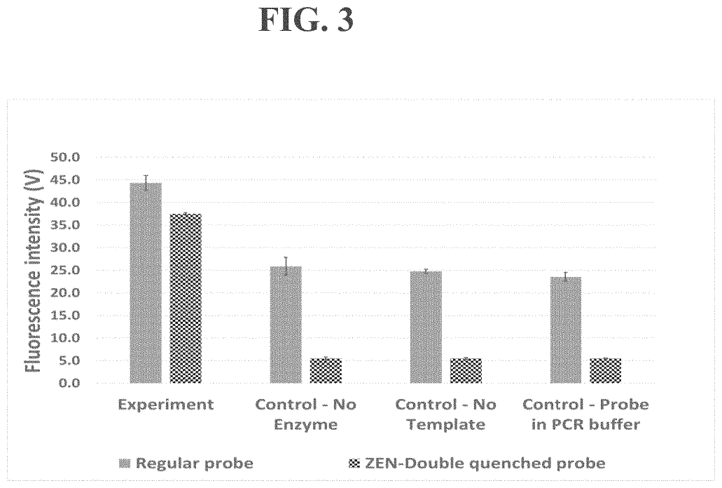

The present inventors have shown that the combined use of a double quenched probe and MMB in a TaqMan™ PCR significantly improves the detection of a target sequence (FIG. 3).

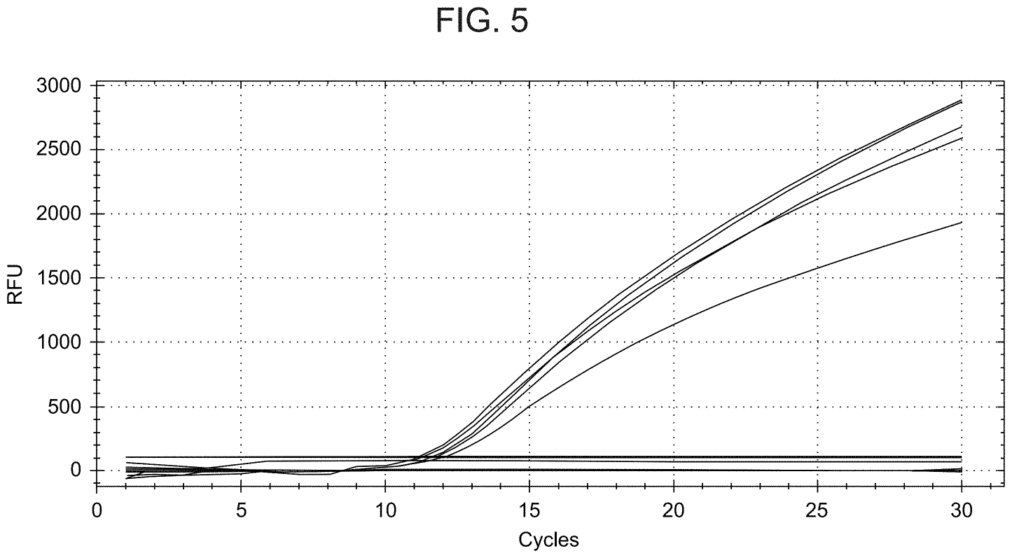

Whilst further reducing the present invention to practice, the present inventors showed that implementation of double quenched probes allows for the reduction in the number of PCR cycles required for reliable detection of female-specific repetitive XhoI DNA sequence to 12 cycles (FIG. 5) using standard Real-Time PCR device and to 6 cycles when using the MMB as a detection system.

Theoretically, MMB-based detection does not require full amplification of the target, only partial degradation of the probe is necessary. Therefore, the length of the cycle can be reduced to a mere 25-30 seconds, allowing the PCR step of the process to be finished in less than 5 minutes.

Whilst further reducing the present invention to practice, the present inventors showed that implementation of double quenched probes allows for the reduction in the number of PCR cycles required for reliable detection of only 10 copies of SARS-CoV2 sequence to 35 cycles when using the MMB as a detection system. This was achieved in a time frame of approximately 35 minutes or less.

The presently disclosed method for detecting SARS-CoV2 infection can be carried out on large groups of people in different scenarios. For example, such methodology may be instrumental in mass-transport hubs (airports, train stations, borders, etc). One such option is the pre-flight or pre-sail screening of the crew and the passengers before boarding—to assure the safety of the maritime and air travel.

Taken together, according to this invention, the combination of a FRET-based assay, double quencher molecules and a detector system such as Magnetic Modulation Biosensing can be used to speed up the time of analyzing DNA sequences, such that commercial testing of DNA for a myriad of purposes including virus detection and chick sexing is now envisaged.

Thus, according to a first aspect of the present invention, there is provided an oligonucleotide probe being 10-300 nucleotides in length, the probe comprising:
(i) a fluorescent moiety attached to a first nucleotide of the probe;
(ii) a first quencher moiety attached to a second nucleotide of the probe;
(iii) a second quencher moiety attached to a third nucleotide which is between the first and the second nucleotide; and
(iv) a first member of an affinity pair attached to the first nucleotide, or to a nucleotide that is 5′ to the third nucleotide.

As used herein, the term “polynucleotide” refers to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The polynucleotide may be single- or double-stranded. A polynucleotide has two opposite ends, the 5′ end and the 3′ end. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′ end or the 5′ end. A polynucleotide sequence, even if internal to a larger polynucleotide (e.g., a sequence region within a polynucleotide), also can be said to have 5′ and 3′ ends.

As used herein, the term “oligonucleotide” refers to a short polynucleotide, typically less than or equal to 300 nucleotides long (e.g., between 5 and 300, preferably between 10 to 200, more preferably between 10 to 100, more preferably between 15 to 50 nucleotides in length and even more preferably between 15 to 30 nucleotides in length). The oligonucleotide of this aspect of the present invention is capable of hybridizing to other polynucleotides, therefore serving as a probe for polynucleotide detection.

Oligonucleotides designed according to the teachings of some embodiments of the invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotides of some embodiments of the invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder.

Specific examples of preferred oligonucleotides useful according to some embodiments of the invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439.

Other oligonucleotides which can be used according to some embodiments of the invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in some embodiments of the invention are disclosed in U.S. Pat. No. 6,303,374.

Oligonucleotides of some embodiments of the invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

The oligonucleotide probe of the invention comprises a polynucleotide sequence, a fluorescent moiety, at least two quencher moieties and a member of an affinity pair. The fluorescent moiety (i.e., fluorophore) and the quenchers may be attached to any nucleotide of the oligonucleotide sequence, so long as the fluorophore and quencher are in close proximity such that FRET results in quenching of the fluorophore. In one embodiment, the fluorophore and the first quencher are attached to the two terminal nucleotides at the opposite ends of the oligonucleotide probe. In one embodiment, the fluorophore is linked close to or at the 5′ end, e.g., the 5′ terminal nucleotide, of the polynucleotide, and the first quencher moiety is linked close to or at the 3′ end, e.g., the 3′ terminal nucleotide, of the oligonucleotide. In another embodiment, the fluorophore is linked close to or at the 3′ end, e.g., the 3′ terminal nucleotide, of the oligonucleotide and the first quencher moiety is linked close to or at the 5′ end, e.g., the 5′ terminal nucleotide, of the oligonucleotide.

In one embodiment, the fluorophore and the second quencher are attached to two nucleotides of the polynucleotide sequence that are between 5 and 60 nucleotides apart. In another embodiment, the fluorophore and the second quencher are attached to two nucleotides of the polynucleotide sequence that are between 5 and 25 nucleotides apart. In still another embodiment, the fluorophore and the second quencher are attached to two nucleotides of the polynucleotide sequence that are between 5 and 20 nucleotides apart. In yet another embodiment, the fluorophore and the second quencher are attached to two nucleotides of the polynucleotide sequence that are between 5 and 15 nucleotides apart. In still another embodiment, the fluorophore and the second quencher are attached to two nucleotides of the polynucleotide sequence that are between 5 and 10 nucleotides apart.

The individual components of the oligonucleotide probe will now be described in detail.

Probe sequence: The sequence of the oligonucleotide probe may be selected such that it is capable of hybridizing to a target polynucleotide under PCR conditions.

As used herein, the term “hybridization” refers to the pairing of complementary, including partially complementary polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

In some embodiments, hybridization may occur despite some degree of mismatches. Usually, hybridization occurs in two polynucleotides having at least 85%, 90%, 95%, 96%, 97%, 98%, or 99% complementarity to each other.

The target polynucleotide that can be detected according to embodiments of the present invention may be derived from any organism, for example, human, Protists (Trichomonas), viruses (e.g., Adenovirus, Herpes viruses, Pox viruses, Retroviruses, (such as Human Immunodeficiency Virus (HIV)), Hepatitis virus (such as Hepatitis A, B, and C), and Papilloma virus), Coronaviruses (e.g. severe acute respiratory syndrome coronavirus 1 (SARS-CoV-1), severe acute respiratory syndrome coronavirus 2 (SARS-CoV-2) or Middle East respiratory syndrome coronavirus (MERS-CoV), bacteria (e.g., Corynebacteria, Pneumococci, Streptococci, Staphylococci, Neisseria, Enterobacteriaciae, Coliform, Salmonellae, Shigellae, other enteric bacilli, hemophilus-Bordetella, Pateurellae, Brucellae, Aerobic Spore-forming Bacilli, Anaerobic Spore-forming Bacilli, Mycobacteria, Actinomycetes, Spirochetes, Mycoplasmas, Rickettsiae, Chlamydia), fungi (e.g., Cryptococcus, Blastomyces, Hisoplasma, Coccidioides, Paracoccidioides, and Candida), and plant, insect or animal cells.

Preferably, the target polynucleotide sequence is repeated in the chromosome at least twice, at least 10 times, at least 100 times, at least 1000 times, at least 10,000 times. The target nucleic acid may be of any length. For example, the target nucleic acid of the present invention contains a known sequence of at least 10 nucleotides, at least 20 nucleotides, 50 nucleotides, at least 100 or more nucleotides, for example, 500 or more nucleotides.

According to a particular embodiment, the target polynucleotide is a sequence that is specific to the W chromosome of a bird (e.g. chicken). In a particular embodiment, the sequence is the Xho I sequence as set forth in SEQ ID NO: 4 or SEQ ID NO: 5. Exemplary primers and probes that can be used to identify the Xho1 sequence are set forth in SEQ ID Nos: 1-3. Exemplary primers and probes that can be used to identify the EcoRI female repetitive sequence are set forth in SEQ ID Nos: 6-8.

According to another embodiment, the target polynucleotide is the E-gene of SARS-CoV2-SEQ ID NO: 17. Exemplary primers that can be used to identify the E gene are set forth in SEQ ID Nos: 9-10. The nucleic acid sequence of the probe is set forth in SEQ ID NO: 11. In a particular embodiment, the probe comprises a sequence as set forth in SEQ ID NO: 16, a fluorescent moiety (e.g. Atto532) a member of an affinity pair (e.g. biotin moiety) and two quenchers, examples of each being further described herein below.

According to a another embodiment, the target polynucleotide is the E-gene of RdRp-gene-SEQ ID NO: 18. Exemplary primers that can be used to identify the RdRp-gene are set forth in SEQ ID Nos: 12-13. The nucleic acid sequence of the probe is set forth in SEQ ID NO: 14. In a particular embodiment, the probe comprises a sequence as set forth in SEQ ID NO: 15, a fluorescent moiety (e.g. Atto532) a member of an affinity pair (e.g. biotin moiety) and two quenchers, examples of each being further described herein below.

The present invention contemplates kits comprising the primer pairs and the associated probe for detecting particular genes.

Fluorescent moiety: Fluorescent moieties (also referred to as fluorophores) are chemical compounds that absorbs light energy at one wavelength and nearly instantaneously emits light at another, longer wavelength of lower energy. The fluorescent moiety of the oligonucleotide probes of the present invention may be compounds that produce chemiluminescence when excited by chemical reaction. Most fluorescent moieties are either heterolytic or polyaromatic hydrocarbons. The fluorescence signature of each individual fluorescent moiety is unique in that it provides the wavelengths and amount of light absorbed and emitted. During fluorescence, the absorption of light excites electrons to a higher electronic state where they remain for about 1-10×10⁻⁸ seconds and then they return to the ground state by emitting a photon of energy. When a population of fluorophores is excited by light of an appropriate wavelength, fluorescent light is emitted. The light intensity can be measured by flurometer or a pixel-by-pixel digital image of the sample.

Fluorescence intensity depends on the efficiency with which fluorescent moieties absorb and emit photons, and their ability to undergo repeated excitation/emission cycles. The intensity of the emitted fluorescent light is a linear function of the amount of fluorophores present. The signal becomes nonlinear at very high fluorophore concentrations.

A wide variety of fluorophores can be used in the oligonucleotide probe of the present invention, including but not limited to: CAL Fluor® Gold 540, CAL Fluor® Orange 560, Quasar® 670, Quasar® 705, 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3, 6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1-sulfonic acid); DABCYL (4-((4-(dimethylamino)phenyl) azo)benzoic acid) Cy5 (Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), ROX, as well as suitable derivatives thereof. Additional examples include, but are not limited to fluorescein, fluorescein chlorotriazinyl, rhodamine green, rhodamine red, tetramethylrhodamine, FITC, Oregon green, Alexa Fluor, FAM, JOE, HEX, Texas Red, TET, TRITC, cyanine-based dye and thiadicarbocyanine dye. According to a particular embodiment, the fluorophore is an Atto dye (for example Atto 655 or Atto 647N).

Quencher moiety: The quencher moiety used in the oligonucleotide probe of this aspect of the present invention can be any material that can quench detectable emission of radiation, for example, fluorescent or luminescent. Quenching can involve any type of energy transfer, including but not limited to, photoelectron transfer, proton coupled electron transfer, dimer formation between closely situated fluorophores, transient excited state interactions, collisional quenching, or formation of non-fluorescent ground state species.

Preferably, the quencher moieties and the fluorescent moiety are attached to the oligonucleotide probe in a configuration that permits energy transfer from the fluorescent moiety to the quencher moieties to result in a reduction of the fluorescence by FRET, as further described herein below. It is not intended that that the term “quencher moiety” be limited to one that participates in FRET.

The degree of the reduction of fluorescence of a fluorescent moiety by the quencher moiety (quenching) is not limited, per se, except that a quenching effect should minimally be detectable by whatever detection instrumentation is used. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more.

The quencher moieties used in the invention may or may not emit fluorescence themselves upon energy transfer from the fluorophore. Some quencher moieties, for example, tetramethyl-6-carboxyrhodamine (TAMRA), can re-emit the energy absorbed from the fluorophore at a wavelength or using a signal type that is also detectable but distinguishable from the fluorophore emission. Other quencher moieties, such as the Black Hole Quenchers (BHQs), including Black Hole Quencher-1 (BHQ-1), Black Hole Quencher-2 (BHQ-2), Black Hole Quencher-3 (BHQ-3) have no native fluorescence, thus can virtually eliminate background problems seen with other quencher moieties. The Black Hole Quenchers, which can be used to quench almost all fluorophores, are commercially available, for example, from Biosearch Technologies, Inc. (Novato, Calif.).

In one embodiment, the first quencher moiety is selected from the group consisting of Dabcyl, TAMRA, Eclipse, DDQ, QSY, Blackberry Quencher, Black Hole Quencher, Qxl, Iowa black FQ, Iowa black RQ, and IRDye QC-1.

In another embodiment, the second quencher moiety is ZEN™ quencher or a TAO™ quencher.

An exemplary probe includes a ZEN internal quencher with 5′ Alexa532, FAM, TET™ HEX™, MAX™, or JOE fluorophores, and a 3′ IBFQ quencher.

Member of an affinity pair: The term “member of an affinity pair” as used herein refers to any component that has an affinity for another component termed here as “second member of the affinity pair.” The binding of the first member to the second member forms an affinity pair between the two components. For example, such affinity pairs include, inter alia, biotin with avidin/streptavidin, antigens or haptens with antibodies, heavy metal derivatives with thiogroups, various polynucleotides such as homopolynucleotides as poly dG with poly dC, poly dA with poly dT and poly dA with poly U. Suitable affinity pairs are also found among ligands and conjugates used in immunological methods. The preferred affinity for use in the present invention is the biotin/streptavidin affinity pair.

In one embodiment, the member of the affinity pair is attached to the same nucleotide as the fluorescent moiety.

In another embodiment, the member of the affinity pair is no more than 1, 2, 3, 4, 5, 6, 7, 8, 9 or even 10 nucleotides away from that which is attached to the fluorescent moiety. Preferably, the member of the affinity pair is not attached to a nucleotide that is 3′ to the nucleotide to which the second quencher moiety is attached.

As mentioned, the oligonucleotide probe of this aspect of the present invention is used to detect the presence of a target nucleic acid sequence.

Thus, according to another aspect of the present invention, there is provided a method of detecting a target nucleic acid sequence in a DNA sample comprising:

(a) contacting the sample with a pair of amplification primers, a polymerase enzyme having 5′ nuclease activity and the oligonucleotide described herein, wherein the oligonucleotide probe anneals within the target nucleic acid sequence bound by said amplification primers, wherein the contacting is effected under conditions that allow extension of the amplification primers and further which allow the 5′ nuclease activity of the polymerase enzyme to cleave the annealed oligonucleotide so as to generate a cleaved fluorescent product;

(b) immobilizing said cleaved fluorescent product via a second member of said affinity pair; and

(c) detecting fluorescence of said cleaved fluorescent product.

DNA may be isolated from the blood immediately or within 1 hour, 2 hours, 3 hours, 4 hours, 5 hours or 6 hours. Optionally the blood is stored at temperatures such as 4° C., or at −20° C. prior to isolation of the DNA. In some embodiments, a portion of the blood sample is used in accordance with the invention at a first instance of time whereas one or more remaining portions of the blood sample (or fractions thereof) are stored for a period of time for later use.

According to one embodiment, the DNA is cellular DNA (i.e. comprised in a cell).

According to still another embodiment, the DNA is comprised in a shedded cell or non-intact cell.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual”, 1989, 2^{nd} Ed., Cold Spring Harbour Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300).

There are also numerous versatile kits that can be used to extract DNA from tissues and bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

According to another embodiment, the DNA is cell-free DNA. For this method, cell lysis is not performed on the sample. Methods of isolating cell-free DNA from body fluids are also known in the art. For example Qiaquick kit, manufactured by Qiagen may be used to extract cell-free DNA from plasma or serum.

According to another embodiment, the DNA sample is cDNA and is derived from an RNA sample. The RNA may be purified from a sample derived from a subject and then subjected to reverse transcription (using a reverse transcriptase enzyme) to generate DNA.

For synthesis of cDNA, template mRNA may be obtained directly from lysed cells or may be purified from a total RNA sample. The total RNA sample may be subjected to a force to encourage shearing of the RNA molecules such that the average size of each of the RNA molecules is between 100-300 nucleotides, e.g. about 200 nucleotides. To separate the heterogeneous population of mRNA from the majority of the RNA found in the cell, various technologies may be used which are based on the use of oligo(dT) oligonucleotides attached to a solid support. Examples of such oligo(dT) oligonucleotides include: oligo(dT) cellulose/spin columns, oligo(dT)/magnetic beads, and oligo(dT) oligonucleotide coated plates.

Generation of cDNA from RNA requires synthesis of an intermediate RNA-DNA hybrid. For this, a primer is required that hybridizes to the 3′ end of the RNA. Annealing temperature and timing are determined both by the efficiency with which the primer is expected to anneal to a template and the degree of mismatch that is to be tolerated.

According to a specific embodiment, the primer comprises a polydT oligonucleotide sequence.

Preferably, the polydT sequence comprises at least 5 nucleotides. According to another is between about 5 to 50 nucleotides, more preferably between about 5-25 nucleotides, and even more preferably between about 12 to 14 nucleotides.

According to another embodiment, the primer which is used to generate cDNA is a sequence-specific primer (e.g. one of the same primers that is used to amplify the cDNA).

Once the DNA is obtained (and optionally purified), the target sequence can be detected by polymerase chain reaction (PCR).

As used herein, “polymerase chain reaction” or “PCR” refers to an in vitro method for amplifying a specific polynucleotide template sequence.

In one embodiment, the PCR reaction involves a repetitive series of temperature cycles and is typically performed in a volume of 20-100 μL. The reaction mix comprises dNTPs (each of the four deoxynucleotides dATP, dCTP, dGTP, and dTTP), amplification primers, buffers, DNA polymerase, and polynucleotide template. One PCR reaction may consist of 5 to 100 “cycles” of denaturation and synthesis of a polynucleotide molecule.

In another embodiment, the PCR amplification reaction is an isothermal amplification reaction. For this embodiment, additional primers are added to the reaction. Such primers do not amplify the DNA but rather serve to protect the DNA during the amplification process.

As used herein, the term “amplification primer” or “amplification primers” refers to an oligonucleotide or oligonucleotides having a sequence complementary to a DNA sequence used as template DNA in PCR reactions. Primers are annealed to a complementary region of the template DNA and extended along the template DNA by a polymerase. The complementary portion of a primer can be any length that supports specific and stable hybridization between the primer and the target sequence under the reaction conditions. The primers used in the invention may have also one or more modified nucleotides that contain modifications to the base, sugar and/or phosphate moieties.

The enzyme used in the PCR reaction of this aspect of the present invention typically has 5′ nuclease activity—for example 5′ to 3′ exonuclease activity (i.e. is capable of removing mononucleotides or oligonucleotides from the 5′ end of a polynucleotide in a sequential manner, (i.e., E. coli DNA polymerase I has this activity whereas the Klenow (Klenow et al, 1970, Proc. Natl. Acad. Sci., USA, 65: 168) fragment does not, (Klenow et al, 1971, Eur. J. Biochem., 22:371)), or polynucleotides are removed from the 5′ end by an endonucleolytic activity that may be inherently present in a 5′ to 3′ exonuclease activity.

As mentioned the assay for detecting DNA sequences relies on sequence-specific oligonucleotide probes that bind to the target nucleic acid sequences specifically. The oligonucleotide probe used in this approach typically contains a fluorescent moeity (the fluorophore) and two quencher moieties, as described herein above. The quencher moieties accept energy from the fluorophore and dissipates it by either proximal quenching or by Förster Resonance Energy Transfer (FRET).

FRET is a distance-dependent interaction between two molecules where the excited donor molecule (e.g., a fluorescent moiety) transfers energy to an acceptor molecule (e.g., a quencher moiety). The energy transfer occurs without emission of photons, and is based on dipole-dipole interactions between the two molecules. For FRET to occur, the emission spectrum of the donor molecule must overlap the absorption spectrum of the acceptor molecule; the transition dipole orientations of the donor and acceptor molecules must be approximately parallel; and the donor and acceptor molecule must be in close proximity to each other (typically 10-100 A). If the donor and acceptor are spaced apart by too great a distance, then the donor fluorophore cannot transfer resonance energy to the acceptor. One type of FRET involves a quencher moiety as the acceptor molecule, the emission spectra of which overlaps with the fluorescent donor molecule. The energy transfer between the two substantially diminished the fluorescence of the donor molecule, a phenomenon known as “quenching”. The efficiency of quenching is directly correlated with the distance between the donor (the fluorophore) and the acceptor (the quencher moiety). As stated above, the quencher moiety itself may or may not be a molecule that can emit fluorescence.

It is preferable that the fluorophore-quencher moiety pair to have sufficient spectral overlap in order to achieve sufficient quenching. Fluorophores with an emission maximum between 500 and 550 nm, such as FAM, TET and HEX, are best quenched by quenchers with absorption maxima between 450 and 550 nm, such as DABCYL and BHQ-1. Fluorophores with an emission maximum above 550 nm, such as rhodamines (including TMR, ROX and Texas red) and Cy dyes (including Cy3 and Cy5) are best quenched by quenchers with absorption maxima above 550 nm (including BHQ-2).

In contrast to traditional probe-based RT-PCRs, where FRET only occurs between the fluorophore and a single quencher moiety, PCRs using the invention employ an additional FRET, which occurs between the fluorophore on the oligonucleotide probe and a second quencher moiety on the oligonucleotide probe. The energy of the fluorophore thus is transferred not only to the first quencher moiety on the oligonucleotide probe, but also to the second quencher moiety of the oligonucleotide probe. The “double quenching” effectively reduces the baseline fluorescence and increases assay sensitivity. The configurations of various oligonucleotide probes and quencher oligonucleotides—which are designed to engage the double quenching—are described below.

The additional quenching does not compromise detection of the fluorescence produced due to the amplification of the template DNA in the PCR reaction; the cleavage of the oligonucleotide probe by the polymerase during the process breaks the proximity between the fluorophore and both quencher moieties, thus allowing unquenched emission of fluorescence.

One of the probe-based quantitation methods employs the 5′ exonuclease activity of polymerases, such as Taq. An oligonucleotide probe that is complementary to the PCR product comprising the target nucleic acid sequence, yet distinct from the PCR amplification primers is labelled with a FRET pair comprising a fluorescent moiety and at least two quencher moieties. The quenchers and the fluorescent moiety are situated within close proximity to each other such that the fluorescence from the fluorescent moiety is quenched by the quencher moieties. During PCR amplification, the 5′ exonuclease proceeds to digest the probe, separating the FRET pair and leading to increased fluorescence. A variation on this technology uses a nucleic acid probe wherein the internal quenching moiety is a hairpin conformation. Another variety on this technology uses a distinct oligonucleotide (referred to herein as a quencher oligonucleotide which comprises the second quenching moiety. This technology is described in WO2017044651, the contents of which are incorporated herein by reference).

Upon hybridization to a sequence of interest, the FRET pair is separated and the donor molecule emits fluorescence.

Alternative methods of analysing the target sequence involves the use of a mismatch repair enzyme such as TDG or MutY using a method described in US patent application No. 20120135873, the contents of which are incorporated herein by reference.

Additional methods for detecting/analysing the target sequence are disclosed in U.S. Pat. No. 9,575,068, the contents of which are incorporated herein by reference.

As mentioned, the method of this aspect of the present invention further includes immobilizing (i.e. capturing) the cleaved fluorescent probe via a second member of the affinity pair.

The second member of the affinity pair is typically attached to a solid support.

Non-limiting exemplary solid supports include polymers (such as agarose, sepharose, cellulose, nitrocellulose, alginate, Teflon, latex, acrylamide, nylon, plastic, polystyrene, silicone, etc.), glass, silica, ceramics, and metals. Such solid supports may take any form, such as particles (including microparticles), sheets, dip-sticks, gels, filters, membranes, microfiber strips, tubes, wells, plates (such as microplates, including 6-well plates, 24-well plates, 96-well plates, 384-well plates, etc.), fibers, capillaries, combs, pipette tips, microarray chips, etc. In some embodiments, the second member of the affinity pair (e.g. biotin-binding moiety) is associated with the surface of a solid support. In some embodiments, the surface of the solid support comprises an irregular surface, such as a porous, particulate, fibrous, webbed, or sintered surface.

In some embodiments, a solid support is selected from a microplate, a microarray chip, and a microparticle. In some embodiments, a solid support is at least partially composed of a polymer. In some embodiments, a microparticle solid support comprises monodisperse or polydisperse spherical beads. Monodisperse microparticles are substantially uniform in size (i.e., they have a diameter standard deviation of less than 5%), while polydisperse microparticles vary in size. In some embodiments, microparticles are composed of the same polymer throughout, or are core-shell polymers, in which the core of the microparticle is composed of one polymer, and the outer layer (or “shell”) is composed of another. In some embodiments, microparticles are magnetic.

M280 Superparamagnetic beads (ThermoFisher Sci. Waltham, Mass., USA) is an example of a commercially available solid support attached to streptavidin.

In some embodiments, the second member of the affinity pair (e.g. a biotin-binding moiety) is attached to a solid support through an amino or sulfhydryl group of the biotin-binding moiety. In some such embodiments, the surface of the solid support comprises a group capable of reacting with a free amine or sulfhydryl group. Nonlimiting exemplary such groups include carboxy, active halogen, activated 2-substituted ethylsulfonyl, activated 2-substituted ethyl carbonyl, active ester, vinylsulfonyl, vinylcarbonyl, aldehyde, epoxy, etc. Some such groups may require the use of an additional reactant to render the group capable of reacting with a free amine or sulfhydryl group. Nonlimiting exemplary additional reactants include cyanogen bromide, carbonyldiimidazole, glutaraldehyde, hydroxylsuccinimide, tosyl chloride, etc.

Many solid supports are known in the art, and one skilled in the art can select a suitable solid support according to the intended application. Similarly, if the solid support is not commercially available with the second member of the affinity pair attached to its surface, one skilled in the art can select a suitable method of attaching the second member of the affinity pair to a solid surface. Exemplary such methods are described, e.g., in U.S. Publication No. US 2008/022004 A1.

Typically, the solid support (attached to the second member of the affinity pair) is added to the PCR reaction after a single cycle, two cycles, three cycles, four cycles, five cycles, six cycles, seven cycles, eight cycles, nine cycles, 10 cycles or more of amplification have taken place. In one embodiment, the solid support is added after completion of at least one isothermal amplification reaction. It will be appreciated that the present inventors also contemplate adding the solid support prior to the PCR reaction.

Thus, for example the amplification part of the method may be effected in less than 10 minutes or even less than 5 minutes. The second member of the affinity pair is contacted with the cleaved probe of the PCR reaction for an amount of time which ensures binding between it and the first member of the affinity pair—e.g. at least 1 minute, more preferably 5 minutes, 10 minutes, or even 15 minutes.

In the case where the solid support is magnetic, e.g. magnetic beads, the solid support may be concentrated using a magnet (see for example Burg et al., Appl. Phys. Lett. 115, 103702 (2019); doi: 10.1063/1.5108891).

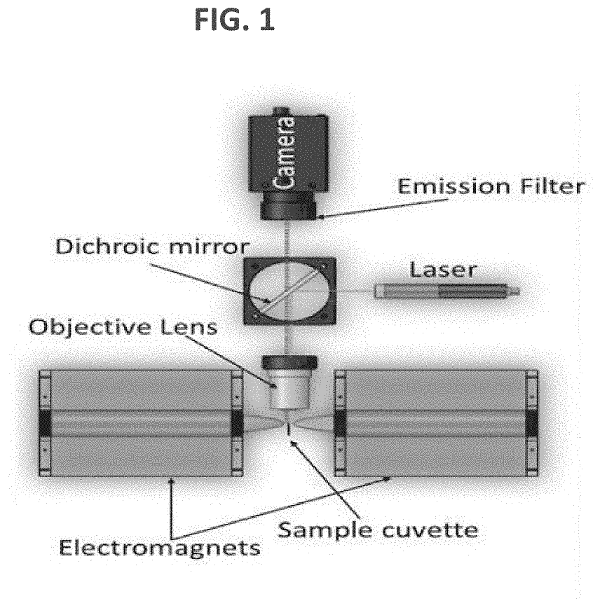

In a particular embodiment, an external oscillating magnetic field gradient may be applied to the sample in order to move the beads. Two external electromagnetic poles may be used to condense the magnetic beads into the detection area and set them in a 1-D periodic motion by modulating the magnetic field gradient—see FIG. 1. This periodic motion, in and out of an orthogonal laser beam, produces a periodic fluorescent light, which is collected by a detector (e.g, a camera, a photomultiplier (PMT), etc.) and demodulated (e.g., using a lock-in amplifier). This system (referred to as magnetic modulation biosensing (MMB) is described in full in Danielli, A et al., Current Pharmaceutical Biotechnology, 2010, 11, 128-137; Danielli, A et al, Opt. Express., 2008, 16, 19253-19259; and Danielli, A Biosens. Bioelectron., 2009, 25,858-863. The magnetic field may serve to concentrate the cleaved fluorescent product so that it is easier to detect.

In one embodiment, the cleaved fluorescent product is detected using flow cytometry (e.g. Luminex).

The fluorescence signal is directly proportional to DNA concentrations over a broad range, and the linear correlation between the PCR product and fluorescence intensity is used to calculate the amount of template DNA (comprising the target nucleic acid sequence) present at the beginning of the reaction. The point at which fluorescence is first detected as statistically significant above the baseline or background, is called the threshold cycle or Ct Value. The Ct Value is the most important parameter for quantitative PCR. This threshold must be established to quantify the amount of DNA in the samples. It is inversely correlated to the logarithm of the initial copy number. The threshold should be set above the amplification baseline and within the exponential increase phase (which looks linear in the log phase). Most assay systems automatically calculate the threshold level of fluorescence signal by determining the baseline (background) average signal and setting a threshold 10-fold higher than this average.

It will be appreciated that the components used to carry out the method of the present invention may be comprised in a diagnostic kit/article of manufacture preferably along with appropriate instructions for use and labels indicating FDA approval for use in diagnosing and/or assessing DNA. Such a kit can include, for example, at least one container including at least one of the above described diagnostic agents (e.g., the oligonucleotide probe described herein) and the solid support attached to the second member of the affinity pair packed in another container. The kit may also include appropriate buffers and preservatives for improving the shelf-life of the kit. The kit may further comprise other components of the reaction such as nucleotides, the necessary primers and the polymerase enzyme.

According to a particular embodiment, the methods of analysing DNA described herein may be carried out in order to diagnose diseases associated with viruses (e.g. RNA viruses, such as coronaviruses).

Thus, according to another aspect of the present invention there is provided a method of diagnosing a disease associated with a coronavirus infection in a subject comprising:
(a) contacting a sample of the subject with:
   (i) a pair of amplification primers, said primers having a sequence such that they are capable of amplifying a target sequence which is specific to the coronavirus;
   (ii) a polymerase enzyme having 5′ nuclease activity; and
   (iii) an oligonucleotide probe, wherein said oligonucleotide probe is between 10-300 nucleotides in length and comprises a fluorescent moiety attached to a first nucleotide of the probe, a first quencher moiety attached to a second nucleotide of the probe, and a second quencher moiety attached to a third nucleotide which is between said first and said second nucleotide, wherein said oligonucleotide probe anneals within said target sequence bound by said amplification primers,
wherein the contacting is effected under conditions that allow extension of the amplification primers and which allow the 5′ nuclease activity of the polymerase enzyme to cleave the annealed oligonucleotide probe so as to generate a cleaved fluorescent product; and
(b) detecting fluorescence of said cleaved fluorescent product, wherein the presence of said cleaved fluorescent product is indicative of the subject having a disease associated with the coronavirus.

Examples of coronaviruses include: human coronavirus 229E, human coronavirus OC43, SARS-CoV-1, HCoV NL63, HKU1, MERS-CoV and SARS-CoV-2.

According to a particular embodiment, the coronavirus is SARS-CoV-2. The disease associated with SARS-CoV-2 infection is Covid-19.

As used herein, the term “diagnosing” refers to ruling in a disease, classifying a disease or a symptom, determining a severity of the disease, monitoring pathology progression, forecasting an outcome of a pathology and/or prospects of recovery.

As used herein, the term “sample” refers to any biological sample (e.g., tissue culture sample or body fluid/tissue sample) which may comprise or permissive for the virus. Preferably the biological sample refers to body fluids such as whole blood, serum, plasma, cerebrospinal fluid, urine, lymph fluids, various external secretions of the respiratory (e.g., nasal wash sample), intestinal, and genitourinary tracts, tears, saliva, semen, sweat, feces, and milk, as well as white blood cells, malignant tissues, amniotic fluid, and chorionic villi.

According to a particular embodiment, the sample is derived from a saliva sample, nasal nasopharyngeal (NP) specimen, an oropharyngeal (OP) specimen, a nasal mid-turbinate swab, an anterior nares specimen or a nasopharyngeal wash.

According to a specific embodiment, the sample is a saliva sample.

The sample is typically treated prior to analysis. For example, cells may be lysed and RNA extracted. cDNA is typically synthesized from the RNA as described herein above. Saliva samples can be analyzed directly without the need for prior RNA extraction. Only thermal treatment is necessary for viral inactivation and lysis.

The PCR reaction components of this aspect of the present invention and the method of detecting the signal have been described herein above.

Once the subject has been diagnosed with a Coronaviral infection, the subject may be treated in a particular way.

Exemplary managements includes, but is not limited to oxygen therapy, non-invasive ventilation, mechanical ventilation, invasive monitoring, last-resort drug, sedation, intensive care admission, surgical intervention, hospital admittance, anti-viral drug, anti-viral regimen, anti-fungal drug, immune-globulin treatment, glucocorticoid therapy, extracorporeal membrane oxygenation, kidney replacement therapy and isolation.

Exemplary agents that may be used to treat coronavirus (e.g. SARS-CoV-2) infections include an antiviral agent, an IV fluid, a vasopressor, an inotrope and a diuretic.

Secondary infections due to coronavirus can be treated using antibiotic or other antibacterial agents.

As mentioned, the methods of analysing DNA described herein may be carried out in order to determine the sex of a bird (e.g. chicken).

Thus, according to another aspect of the present invention there is provided a method of determining the sex of a chicken comprising:

(a) contacting DNA of the chicken with:
(i) a pair of amplification primers, said primers having a sequence such that they are capable of amplifying a target sequence on a W chromosome that repeats at least 10 times;
(ii) a polymerase enzyme having 5′ nuclease activity; and
(iii) an oligonucleotide probe, wherein said oligonucleotide probe is between 10-300 nucleotides in length and comprises a fluorescent moiety attached to a first nucleotide of the probe, a first quencher moiety attached to a second nucleotide of the probe, and a second quencher moiety attached to a third nucleotide which is between said first and said second nucleotide, wherein said oligonucleotide probe anneals within said target sequence bound by said amplification primers,

wherein the contacting is effected under conditions that allow extension of the amplification primers and which allow the 5′ nuclease activity of the polymerase enzyme to cleave the annealed oligonucleotide probe so as to generate a cleaved fluorescent product; and

(b) detecting fluorescence of said cleaved fluorescent product.

In one embodiment, the method is carried out in ovo, i.e., on non-hatched eggs. A biological sample can be retrieved from an embryo at any stage, including the stage of day 1 wherein the germinal disc is at the blastodermal stage and the segmentation cavity takes on the shape of a dark ring; the stage of day 2 wherein the first groove appears at the center of the blastoderm and the vitelline membrane appears; the stage of day 3 wherein blood circulation starts, the head and trunk can be discerned, as well as the brain and the cardiac structures which begins to beat; the stage of day 4 wherein the amniotic cavity is developing to surround the embryo and the allantoic vesicle appears; the stage of day 5 wherein the embryo takes a C shape and limbs are extending; the stage of day 6 wherein fingers of the upper and lower limbs becomes distinct; the stage of day 7 wherein the neck clearly separates the head from the body, the beak is formed and the brain progressively enters the cephalic region; the stage of day 8 wherein eye pigmentation is readily visible, the wings and legs are differentiated and the external auditory canal is opening; the stage of day 9 wherein claws appears and the first feather follicles are budding; the stage of day 10 wherein the nostrils are present, eyelids grow and the egg-tooth appears; the stage of day 11 wherein the palpebral aperture has an elliptic shape and the embryo has the aspect of a chick; the stage of day 12 wherein feather follicles surround the external auditory meatus and cover the upper eyelid whereas the lower eyelid covers major part of the cornea; the stage of day 13 wherein the allantois becomes the chorioallantoic membrane while claws and leg scales becomes apparent; the stage of days 14 to 16 wherein the whole body grows rapidly, vitellus shrinking accelerates and the egg white progressively disappears; the stage of day 17 wherein the renal system produces urates, the beak points to the air cell and the egg white is fully resorbed; the stage of day 18 wherein the vitellus internalized and the amount of amniotic fluid is reduced; the stage of day 19 wherein vitellus resorption accelerates and the beak is ready to pierce the inner shell membrane; the stage of day 20 wherein the vitellus is fully resorbed, the umbilicus is closed, the chick pierces the inner shell membrane, breathes in the air cell and is ready to hatch; the stage of day 21 wherein the chick pierces the shell in a circular way by means of its egg-tooth, extricates itself from the shell in 12 to 18 hours and lets its down dry off.

More specifically, the method of the invention may be applicable in determining the gender of an avian embryo in-ovo, inside the egg, at every stage of the embryonic developmental process. More specifically, from day 1, from day 2, from day 3, from day 4, from day 5, from day 6, from day 7, from day 8, from day 9, from day 10, from day 11, from day 12, from day 13, from day 14, from day 15, from day 16, from day 17, from day 18, from day 19, from day 20 and from day 21. More specifically, the method of the invention may be applicable for early detection of the embryo's gender, specifically, from day 1 to day 10, more specifically, between days 1 to 5.

Thus, the method of the invention may be applicable for fertilized unhatched eggs.

As used herein, the term “fertilized egg” refers to an egg laid by a hen wherein the hen has been mated by a rooster within two weeks, allowing deposit of male sperm into the female infundibulum and fertilization event to occur upon release of the ovum from the ovary. “Unhatched egg” as used herein, relates to an egg containing an embryo (also referred to herein as a fertile egg) within a structurally integral shell.

DNA may be retrieved from an unhatched egg using a technology similar to one that is used in in-ovo vaccination process—see for example embrexbiodevicesdotcom/About-Embrex/.

It will be appreciated that the method according to this aspect of the present invention may be carried out on birds (e.g. chicks) of any age—preferably before it is possible to determine their sex by physical examination of secondary sexual characteristics. Thus, the chick may be less than 1 day old, 1 day old, two days old, three days old, four days old, five days old, six days old or seven days old.

The PCR reaction components of this aspect of the present invention and the method of detecting the signal have been described herein above.

It is expected that during the life of a patent maturing from this application many relevant fluorescent moieties and quencher moieties will be developed and the scope of these terms is intended to include all such new technologies a priori.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Sample collection: 1.5 ml whole blood samples of adult chickens (male and female) were obtained. Samples were collected using heparin flushed disposable syringes (Heparin lock flush solution, Kamada, Beit Kama, Israel). Immediately after collection, the samples were aliquoted (0.5 ml/tube) and refrigerated on ice. One aliquot of each sample was used for DNA extraction. The rest of the aliquoted samples were frozen at −80° C. until further use.

DNA extraction: 0.5 ml of the adult chickens' whole blood was used as a source of genomic DNA. DNA extraction was performed using QIAGEN QIAamp DNA Blood Maxi Kit (QIAGEN, GmbH, Hilden, Germany) according to manufacturer's protocol.

Bioinformatic analysis: Bioinformatic analysis was performed on the most recent available version of chicken genome (Gallus_gallus-5.0: GCA_000002315.3) [18] using NCBI BLAST+web service. Known female-specific repetitive sequences [16] were verified against the genomic data to determine their relative abundance and chromosomal affiliation. Highly repetitive sequences located within W (female) chromosome (XhoI and RhoI) where chosen as potential targets.

Oligonucleotides: All the oligonucleotides were designed using PrimerQuest Tool web service from Integrated DNA Technologies, Inc (Skokie, Ill., USA) and purchased from the same vendor. Regular and modified Tagman™ assay components (double quenched (“Zen”) probe and primers for corresponding DNA fragment) were received as lyophilized powder and resuspended in DNase-free ultrapure water (Biological industries, Beit haEmek, Israel) to a final concentration of 100 μM. Resulting solutions were aliquoted and frozen until further use. Double quenched probe of 23b, targeting the selected repetitive sequence, was functionalized by attachment of biotin and fluorophore (Alexa532) molecules to the first nucleotide at the 5′ end and dark quencher molecules (ZEN quencher at 9^{th} nucleotide and Iowa Black FQ at the 3′ end). Table 1 summarizes the synthetic oligonucleotides used in the examples.

TABLE 1

| Name of | | |
|---|---|---|
| the oligo- | | |
| nucleotide | Sequence (5′-3′) | Modifications |
| XhoI | CTGCACTTCCTTCCCG | None |
| Forward | AAA (SEQ ID NO: | |
| primer | 1) | |
| XhoI | CGGCTGAAAGGTGGTA | None |
| Reverse | CTT (SEQ ID NO: | |
| primer | 2) | |
| Zen probe | CGCTTCACTCACAAAG | Biotin + Alexa532 |
| | CACGCATT (SEQ ID | at 5′, Zen quencher |
| | NO: 3) | at 9^{th} nucleotide, |
| | | Iowa Black FQ at |
| | | the 3′ |

Magnetic beads: For MMB testing, M280 Superparamagnetic beads (ThermoFisher Sci. Waltham, Mass., USA) were prepared according to manufacturer's instructions and diluted prior to use to a final concentration of 10⁶ beads/ml.

The assay: PCR reactions for magnetic modulation biosensing (MMB) testing were performed using the Mastercycler Nexus Thermal cycler (Eppendorf, Hamburg, Germany). Control qPCR reactions were performed using the CFX96Touch Real-Time PCR detection system (Bio-Rad, Hercules, Calif., USA). Five male and five female genomic DNA samples with final concentration of 50 ng/μl were prepared. Reaction mix was prepared in quadruplicate for each DNA sample with final volume of 25 ul/reaction. Reagents mix for each reaction contained: 50 ng of genomic DNA (50 ng/μl), 12.5 μl of JumpStart Taq ReadyMix (Sigma-Aldrich, MO, USA), 500 pmol of Forward and 500 pmole of Reverse primers, 10 pmol of double quenched Zen probe (0.1 μM) and 0.5 μl of DNase/RNase free ultrapure water, mixed together in standard 200 μl PCR tube. 2 types of control reactions were prepared: in one genomic DNA was substituted by 1 ul of ultrapure water, while in another one JumpStart Taq ReadyMix was substituted with 12.5 μl of ultrapure water. Primer and probe concentrations and hybridization conditions were optimized in advance and final reaction conditions for XhoI target consisted of 2 minutes at 94° C. followed by 5, 10, 20 and 30 cycles of 30 seconds at 94° C., 30 seconds at 54° C. and 30 seconds at 68° C. Following the completion of designated number of cycles—the reaction tubes were removed from the thermocycler and their content was supplemented with 75 μl of ultrapure water for final volume of 100 μl.

Preparation for MMB testing: In the presence of the target sequence, 5′>3′ exonuclease activity of the Taq polymerase causes the degradation of the probes. Physical separation of 5′ nucleotides, conjugated with Biotin and Alexa532 fluorophore, from the quencher(s) leads to significantly higher fluorescence levels—compared to samples that do not contain the target DNA. In these samples, PCR reaction does not happen, probes are left intact and fluorescence stays quenched.

In order to analyze the results using the MMB system, 5′ nucleotides with conjugated functional molecules were attached to magnetic beads. For that purpose, 100 μl of the M280 StreptAvidin (ThermoFisher Sci. Waltham, Mass., USA) superparamagnetic beads solution (˜100,000 beads) were added to PCR reaction tubes in a final volume of 200 μl. Resulting solutions were transferred to 1.5 ml tubes and incubated at room temperature with constant rotation (TR-1550, MRC, Israel, 40 rpm) for 15 minutes. Upon completion of the attachment step, beads were collected using MagJet magnetic rack (ThermoFisher Sci. Waltham, Mass., USA), supernatant was discarded and beads were resuspended in 400 ul PBS×1 buffer (Biological Industries, Beit haEmek, Israel) supplemented with 0.01% (v/v) Tween 20 (Sigma-Aldrich, MO, USA).

MMB system: The MMB system is illustrated in FIG. 1 and previously described in Danielli et al., Opt Express, 2008. 16(23): p. 19253-9. The system is designed to improve the signal-to-noise ratio at low concentrations of a target analyte. In order to do that, MMB utilizes superparamagnetic beads covered with streptavidin molecules (beads with other conjugates are available for different types of biological assays). Biotin molecule conjugated to 5′ nucleotide of the Zen probe, allows attachment of that nucleotide to streptavidin coated magnetic bead. Following the successful attachment of the 5′ nucleotides of the cleaved probes to the bead surface, external oscillating magnetic field is applied to the sample in order to concentrate the beads into compact group and move them from side to side of the testing tube. Laser beam is applied at a fixed position and moving beads cross it, creating a clearly distinguishable flickering pattern. The fluorescence level measured while beads are travelling through the laser beam is proportional to the initial concentration of target DNA fragments and to the number of PCR cycles. MMB system ability to amplify the specific signal and discern it from background noise of intact detection probes, eliminates the need for the washing steps, normally required by other detection methods to get rid of unwanted background fluorescence. As a result, handling time for each tested sample is considerably shorter.

Results

Initial experiments were performed on 50 ng of genomic DNA with female-specific, non-repetitive DNA fragment as a target [Granevitze, Z., et al., Journal of Heredity, 2007. 98(3): p. 238-242].

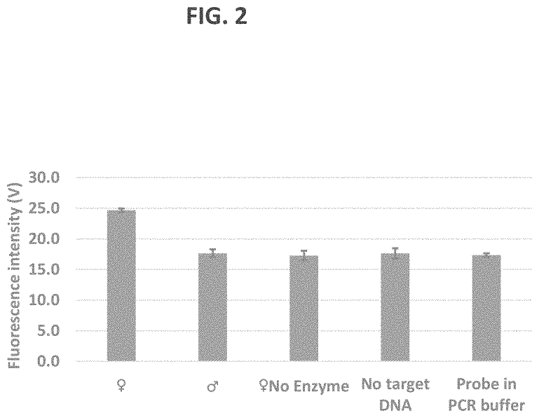

Using conventional Tagman™ assay setup and MMB as a detection system it was possible to discriminate between male and female samples after 30 cycles of PCR, with 40% stronger fluorescent signal in female samples. FIG. 2 depicts the results of the experiment.

Considering the high number of PCR cycles required for reliable detection, the present inventors sought to improve upon these results. One of the reasons for relatively low delta between male and female samples is the high fluorescent background which is identical in all the control samples. Reduction of the background should allow higher detection sensitivity at reduced number of PCR cycles required for female samples identification. To do this, the present inventors used a ZEN quencher located at the 9^{th} base from the fluorophore. FIG. 3 presents the results of comparative experiment with regular and ZEN double quenched probes.

In control samples of this experiment ZEN probe allowed reduction of background fluorescence by ˜80%, when compared to a regular probe, while fluorescence of the sample with target molecule was reduced by ˜15%. This is equivalent to 2.7 times improvement in signal to noise ratio (SNR).

Female W chromosome contains low number of functional genes and high number of repetitive sequences, some of them are found in more than 5000 copies/chromosome, providing “natural amplification” of the potential targets, which can be used to reduce the number of PCR cycles required for detection. Such is the XhoI female-specific repetitive sequence [14, 16]. This sequence is 717 bp long and its repetitions cover significant part of the W chromosome. A Tagman™ assay was designed to target the inner portion of the XhoI fragment. Assay components are presented in Table 1.

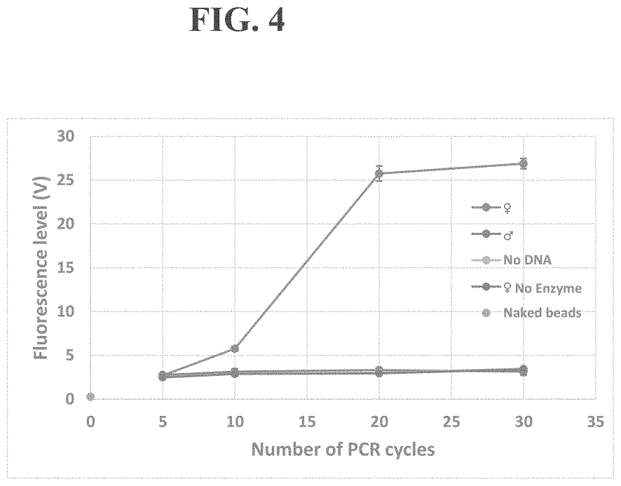

Utilization of the repetitive sequences for reliable discrimination between male and female samples was tested and the results are presented in FIG. 4.

In order to compare the performance of MMB detection system with the conventional Real-Time PCR device (CFX96Touch Bio-Rad, Hercules, Calif., USA), the same experiment with 5 male and 5 female samples was performed. Results of the experiment are presented in FIG. 5.

According to the presented results, use of ZEN probes allows discrimination of male and female samples (based on presence of the XhoI female-specific repetitive fragment) starting from the 6th PCR cycle when using the MMB detection system and starting from the 12th PCR cycle when using the conventional Real-Time PCR instrument.

### Example 2

Currently, rapid and accurate detection of the SARS-CoV2 and diagnosis of the COVID-19 are an urgent and unmet need in almost every country of the world. Given the importance and the urgency of the matter, the present inventors chose to demonstrate the capabilities of the developed method by detecting the SARS-CoV2 infections in clinical samples of patients from the Sheba medical center in Israel.

Materials and Methods

Sample collection: the sample was derived from a nasal nasopharyngeal (NP) specimen, an oropharyngeal (OP) specimen, a nasal mid-turbinate swab, an anterior nares specimen or a nasopharyngeal wash. In addition, 500 μL of saliva samples were obtained from patients and tested using the RT-qPCR (including RNA extraction and purification) and MMB systems (without prior RNA extraction and purification).

Oligonucleotides: All the oligonucleotides were purchased from Integrated DNA Technologies, Inc (Skokie, Ill., USA). All oligonucleotides (double quenched hydrolysis probes and primers for corresponding DNA fragments) were received as lyophilized powder and resuspended in DNase-free ultrapure water (Biological industries, Beit haEmek, Israel) to a final concentration of 100 μM. Resulting solutions were aliquoted and frozen until further use. Double quenched hydrolysis probe of 26 bases, targeting the selected SARS-CoV2-specific viral sequence (E-gene), was functionalized by attachment of biotin and fluorophore (Atto532) molecules to the first nucleotide at the 5′ end and dark quencher molecules (ZEN quencher at 9^{th} nucleotide and Iowa Black FQ at the 3′ end). Table 2 represents the synthetic oligonucleotides used in the SARS-CoV2 detection assay.

TABLE 2

| Name of | | |
|---|---|---|
| the oligo- | | |
| nucleotide | Sequence (5′-3′) | Modifications |
| E-gene | ACAGGTACGTTAATAGTTAA | None |
| Forward | TAGCGT SEQ ID NO: 9 | |
| primer | | |
| E-gene | ATATTGCAGCAGTACGCACA | None |
| Reverse | CA - SEQ ID NO: 10 | |
| primer | | |
| Double- | (Atto532 + Biotin)- | Biotin + Atto532 |
| quenched | ACACTAGCC/ZENQ/ATCCT | at 5′, Zen |
| modified | TACTGCGCTTCG-IAbkFQ - | quencher at 9^{th} |
| hydrolysis | SEQ ID NO: 11 | nucleotide, Iowa |
| probe | | Black FQ at the |
| | | 3′ |
| RdRp-gene | GTGARATGGTCATGTGTGGCG | None |
| Forward | G - SEQ ID NO: 12 | |
| primer | | |
| RdRp-gene | CARATGTTAAASACACTATTA | None |
| Reverse | GCATA - SEQ ID NO: 13 | |
| primer | | |
| Double- | (Atto532 + Biotin) | Biotin + Atto532 |
| quenched | CAGGTGGAA/ZENQ/CCTCAT | at 5′, Zen |
| modified | CAGGAGATGC-IABKFQ - | quencher at 9^{th} |
| hydrolysis | SEQ ID NO: 14 | nucleotide, Iowa |
| probe | | Black FQ at the |
| | | 3′ |

Magnetic beads: For MMB testing, M280 Superparamagnetic beads (ThermoFisher Sci. Waltham, Mass., USA) were prepared according to manufacturer's instructions, photobleached for 18 hours [6] and diluted to final concentration of 10⁶ beads/ml.

The assay: PCR reactions for MMB testing were performed using the RP-96 Thermal cycler (MIULAB, Hangzhou, China). Reaction mixes were prepared in accordance with the 10 required number of samples with a final volume of 20 μL per reaction. Reagents mix for each E-gene detection reaction contained: 5 μL of RNA extract from the patient, 10 μl of SensiFast PCR mix (Bioline, London, UK), 0.2 μL of Reverse Transcriptase enzyme (Bioline, London, UK), 0.4 μL of RNase inhibitor (Bioline, London, UK), 8 pmol (0.8 μL of 10 μM solution) of Forward and 8 pmole of Reverse primers, 4 pmol (0.4 μL of 10 μM solution) of modified double quenched E-gene hydrolysis probe and 2.4 μl of DNase/RNase free ultrapure water, mixed in standard 200 μl PCR tube.

Reagents mix for each RdRp gene detection reaction contained: 5 μL of RNA extract from the patient, 10 μl of SensiFast PCR mix (Bioline, London, UK), 0.2 μL of Reverse Transcriptase enzyme (Bioline, London, UK), 0.4 μL of RNase inhibitor (Bioline, London, UK), 12 pmol (0.8 μL of 10 μM solution) of Forward and 16 pmol of Reverse primers, 4 pmol (0.4 μL of 10 μM solution) of modified double quenched RdRp-gene hydrolysis probe and 1.2 μl of DNase/RNase free ultrapure water, mixed together in standard 200 μl PCR tube.

2 types of control reactions were prepared: in negative control the RNA extract was replaced with 5 μL of ultrapure water, in positive control it was replaced with 5 μL of target solution containing 10 synthetic targets. Primers and probe concentrations and hybridization conditions were optimized in advance and final reaction conditions for the E-gene and RdRp-gene targets were as follows 10 min. at 45° C. followed by 2 min. at 95° C. and 45 cycles of 5 sec. at 94° C., 8 sec. at 58° C., and 8 sec. at 60° C. Following the completion of the PCR phase of the test the reaction tubes were removed from the thermocycler and their content was transferred to 96-wells microplate.

Saliva was tested as a source of the SARS-CoV-2 testing sample. Three samples from healthy patients were tested and compared with the same samples spiked with 10 and 100 copies of a synthetic E-gene target RNA. In addition, 10 and 100 copies of the synthetic E-gene target RNA were spiked in water (Ultra Pure H₂O) and compared with the saliva-based samples. All the samples were tested by the MMB system and the signal was normalized to the signal generated by a negative H₂O only sample.

Finally, two samples were spiked (one of H₂O only and the other of a healthy patient saliva, 4 μL from each sample) with 1 μL of RNA extract from a positive patient (confirmed positive using real time PCR).

Preparation for the MMB testing: In the presence of the target sequence, 5′>3′ exonuclease activity of the Taq polymerase causes the degradation of the probes. Physical separation of 5′ nucleotides, conjugated with Biotin and Atto532 fluorophore, from the quencher(s) leads to significantly higher fluorescence levels—compared to samples that do not contain the target DNA. In these samples, PCR reaction does not happen, probes are left intact and fluorescence stays quenched.

To analyze the results using the MMB system, 5′ nucleotides with conjugated functional molecules are attached to magnetic beads. For that purpose, 25 μl of the photobleached [6] M280 StreptAvidin (ThermoFisher Sci. Waltham, Mass., USA) superparamagnetic beads solution (˜25 000 beads) were added to each of the wells containing the completed PCR reactions. The wells were supplemented with 55 μL of phosphate buffer solution×1 (PBS, HyLabs, Rehovot, Israel) for a final incubation volume of 100 μL/well. The plate was incubated at room temperature with constant mixing (RH-24 3D gyratory rocker, MIULAB, Hangzhou, China) for 5 min. Upon completion of the attachment step, beads were collected by placing the microplate on a Magnetic Stand-96 (ThermoFisher Sci. Waltham, Mass., USA) for 2 minutes, after which the buffer was discarded and replaced with 100 μL of PBS×1 buffer (Biological Industries, Beit haEmek, Israel) supplemented with 0.05% (v/v) Tween 20 (Sigma-Aldrich, MO, USA).

MMB system: As described for Example 1.

### RESULTS

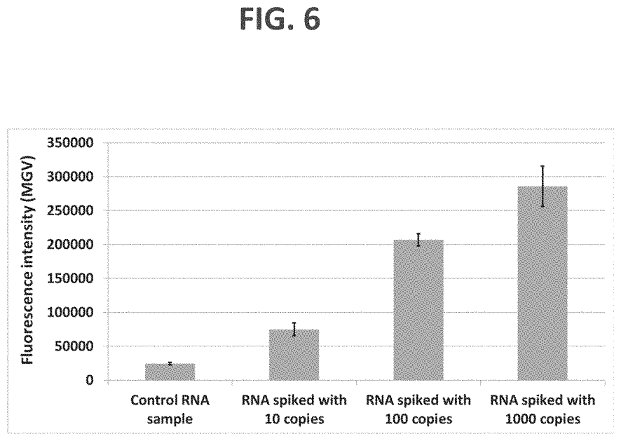

Initial experiments were performed on verified SARS-CoV2-negative RNA extract spiked with synthetic target solutions containing 10, 100, and 1000 copies of the E-gene DNA fragment/reaction. After 35 amplification cycles, the MMB-based detection method allowed a clear distinction between the positive and negative control samples (FIG. 6).

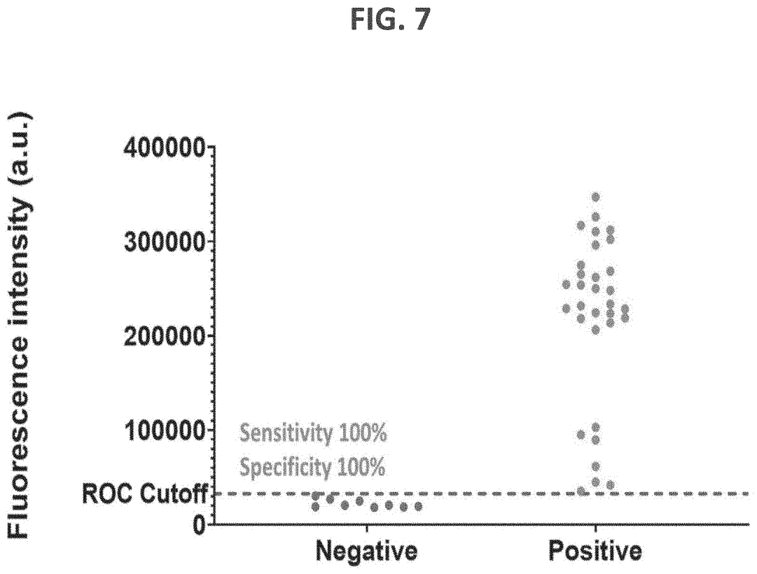

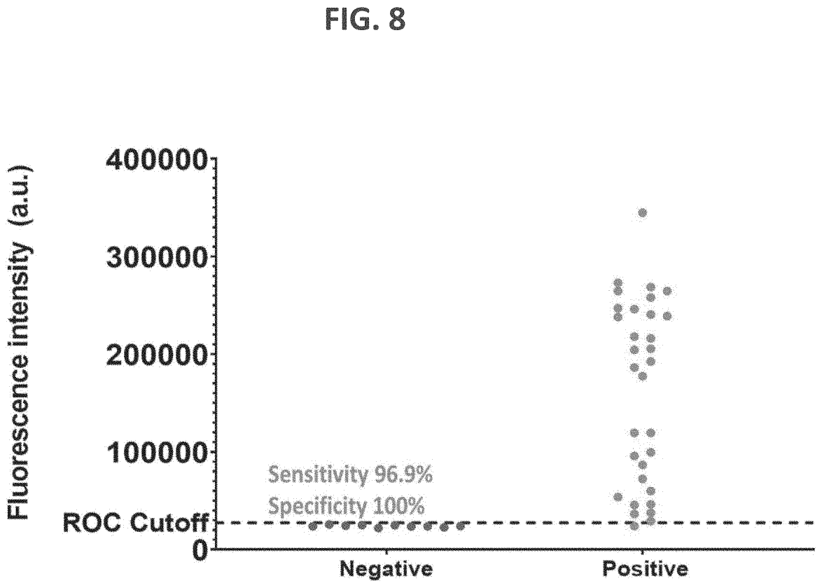

Following the successful initial calibration experiments, the method was implemented on clinical samples. Samples' designation as “positive” or “negative” was verified by the conventional RT-qPCR. Results were analyzed using GraphPad Prism statistical software. Standard receiver operating characteristics (ROC) analysis was performed and the results were presented as a scatter plot with calculated ROC cutoff value (FIG. 7 and FIG. 8).

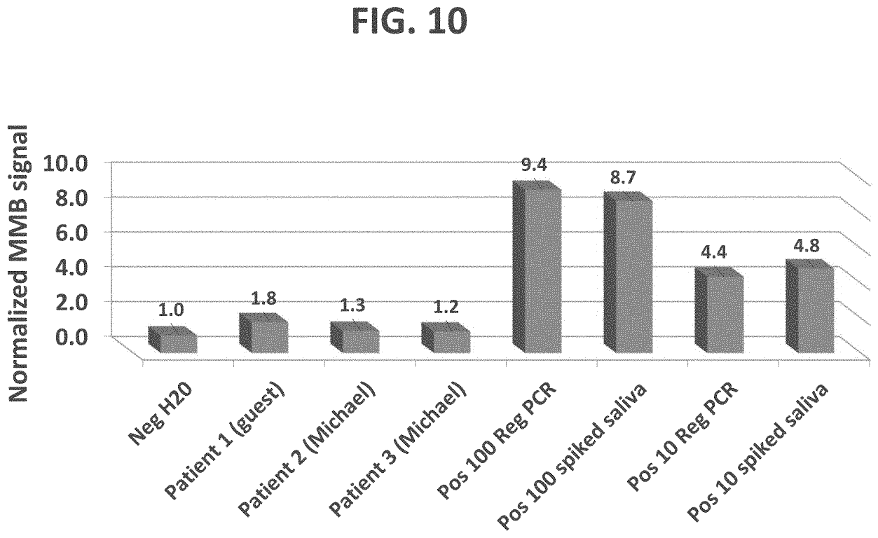

In addition, saliva samples from healthy patients showed minimal increase in background fluorescence (compared with a negative sample of ultra-pure water), while the fluorescent signal from the same samples spiked with 10 or 100 copies of the synthetic E-gene target showed significant increase (3-4 folds and 6-7 folds, respectively) over the healthy patients' signal (FIG. 10).

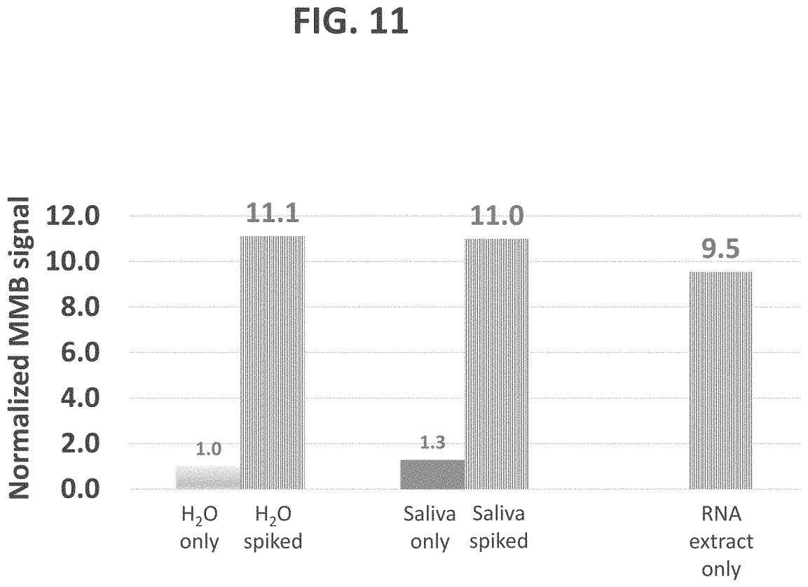

Healthy patient's sample and a negative control sample (ultra-pure water) spiked with 1 μl of a purified RNA extract from a confirmed SARS-CoV-2-positive sample showed comparable signal to the purified RNA extract sample (FIG. 11).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of diagnosing a disease associated with a coronavirus infection in a subject comprising:
(a) contacting a sample of the subject with:
(i) a pair of amplification primers, said primers having a sequence such that they are capable of amplifying a target sequence which is specific to the coronavirus;
(ii) a polymerase enzyme having 5′ nuclease activity; and
(iii) an oligonucleotide probe, wherein said oligonucleotide probe is between 10-300 nucleotides in length and comprises a fluorescent moiety attached to a first nucleotide of the probe, a first quencher moiety attached to a second nucleotide of the probe, and a second quencher moiety attached to a third nucleotide which is between said first and said second nucleotide, wherein said oligonucleotide probe anneals within said target sequence bound by said amplification primers,
wherein the contacting is effected under conditions that allow extension of the amplification primers and which allow the 5′ nuclease activity of the polymerase enzyme to cleave the annealed oligonucleotide probe so as to generate a cleaved fluorescent product; and
(b) detecting fluorescence of said cleaved fluorescent product, wherein the presence of said cleaved fluorescent product is indicative of the subject having a disease associated with the coronavirus.

2. The method of claim 1, wherein said oligonucleotide probe further comprises a first member of an affinity pair attached to said 5′ nucleotide, wherein said first nucleotide of the probe is the 5′ nucleotide and wherein said second nucleotide of the probe is the 3′ nucleotide and wherein the third nucleotide is 5-20 nucleotides from said 5′ nucleotide.

3-5. (canceled)

6. The method of claim 1, further comprising immobilizing said cleaved fluorescent product via a second member of said affinity pair following said contacting and prior to said detecting.

7-9. (canceled)

10. The method of claim 1, wherein said coronavirus is severe acute respiratory syndrome coronavirus 2 (SARS-CoV-2).

11. The method of claim 10, wherein said target sequence is comprised in the E_Sarbeco gene (E-gene) of said SARS-CoV2 virus, the nucleic acid sequence of the probe is as set forth in SEQ ID NO: 16 and the nucleic acid sequence of the primers are as set forth in SEQ ID NOs: 9 and 10.

12-13. (canceled)

14. The method of claim 10, wherein said target sequence is comprised in the RdRp gene of said SARS-CoV2 virus, the nucleic acid sequence of the probe is as set forth in SEQ ID NO: 15 and the nucleic acid sequence of the primers are as set forth in SEQ ID NOs: 12 and 13.

15-28. (canceled)

29. An oligonucleotide probe being 10-300 nucleotides in length, the probe comprising:
(i) a fluorescent moiety attached to a first nucleotide of the probe;
(ii) a first quencher moiety attached to a second nucleotide of the probe;
(iii) a second quencher moiety attached to a third nucleotide which is between said first and said second nucleotide; and
(iv) a first member of an affinity pair attached to said first nucleotide or to a nucleotide that is 5′ to said third nucleotide.

30. The oligonucleotide probe of claim 29, wherein said first nucleotide of the probe is the 5′ nucleotide.

31. The oligonucleotide probe of claim 29, wherein said second nucleotide of the probe is the 3′ nucleotide.

32. The oligonucleotide of claim 29, wherein the third nucleotide is 5-20 nucleotides from said 5′ nucleotide.

33. (canceled)

34. The oligonucleotide of claim 29, wherein said first quencher moiety is selected from the group consisting of Dabcyl, TAMRA, Eclipse, DDQ, QSY, Blackberry Quencher, Black Hole Quencher, Qxl, Iowa black FQ, Iowa black RQ, and IRDye QC-1 and said second quencher moiety is a ZEN™ quencher or a TAO™ quencher.

35. (canceled)

36. The oligonucleotide of claim 29, wherein said affinity pair comprises a biotin/avidin affinity pair or a biotin/streptavidin affinity pair.

37. The oligonucleotide of claim 29, being no longer than 30 nucleotides.

38. The oligonucleotide of claim 29, having a sequence such that it hybridizes to a nucleic acid sequence that repeats more than 100 times in a single chromosome.

39. (canceled)

40. The oligonucleotide of claim 29, comprising a nucleic acid sequence as set forth in SEQ ID NOs: 11, 14, 15 or 16.

41. (canceled)

42. A kit comprising the oligonucleotide of claim 29 and a second member of said affinity pair which is attached to a solid support.

43. A method of detecting a target nucleic acid sequence in a DNA sample comprising:
(a) contacting the sample with a pair of amplification primers, a polymerase enzyme having 5′ nuclease activity and the oligonucleotide of claim 29, wherein the oligonucleotide probe anneals within the target nucleic acid sequence bound by said amplification primers, wherein the contacting is effected under conditions that allow extension of the amplification primers and further which allow the 5′ nuclease activity of the polymerase enzyme to cleave the annealed oligonucleotide so as to generate a cleaved fluorescent product;
(b) immobilizing said cleaved fluorescent product via a second member of said affinity pair; and
(c) detecting fluorescence of said cleaved fluorescent product.

44. The method of claim 43, wherein said second member of said affinity pair is attached to a solid support.

45. The method of claim 44, wherein said solid support comprises a magnetic bead.

46. The method of claim 45, further comprising using a magnetic field to concentrate said cleaved fluorescent product prior to said detecting.

47. (canceled)
